(19) United States
(12) Patent Application Publication
(10) Pub. No.: US 2008/0064877 A2
(43) Pub. Date: Mar. 13, 2008

(54) PROCESS FOR THE PREPARATION OF ROSIGLITAZONE

(75) Inventors: Ernesto Duran Lòpez, L'Hospitalet de Llobregat (ES); Gabriel Tojo Suárez, Ames (ES)

Correspondence Address: ROTHWELL, FIGG, ERNST & MANBECK, P.C., 1425 K STREET, N.W., SUITE 800, WASHINGTON, DC, 20005, us

(73) Assignee: Medichem S.A., Sant Joan Despi (ES)

(21) Appl. No.: 11/579,439

(22) Filed: Nov. 2, 2006

(51) Int. Cl. C07D 417/12

(52) U.S. Cl. 546/269.7

(57) ABSTRACT

The present invention relates to a process for the preparation of 5-{4-[2-(N-methyl-N-(2-pyridyl)amino)ethoxy]benzyl-2,4-thiazolidinedione of formula (I) (Rosiglitazone), which comprises the reaction of 5-{4-[2-(N-methyl-N-(2-pyridyl)amino)ethoxy]benzylidene-2,4-thiazolidinedione of formula (II), with a 1,4-dihydropyridine of general formula (III).

The present invention relates to a process for the preparation of 5-{4-[2-(N-methyl-N-(2-pyridyl)amino)ethoxy]benzyl-2,4-thiazolidinedione of formula (I) (Rosiglitazone), which comprises the reaction of 5-{4-[2-(N-methyl-N-(2-pyridyl)amino)ethoxy]benzylidene-2,4-thiazolidinedione of formula (II), with a 1,4-dihydropyridine of general formula (III).

5-{4-[2-(N-Methyl-N-(2-pyridyl)amino)ethoxy]benzyl-2,4-thiazolidinedione of formula (I), also named Rosiglitazone (its international common denomination), is a known drug for the treatment of non insulin-dependent diabetes mellitus, preferably as its maleate salt.

According to the prior art, Rosiglitazone (I) can be obtained from 5-{4-[2-(N-methyl-N-(2-pyridyl)amino)ethoxy]benzylidene-2,4-thiazolidinedione (II) by treatment under the following reaction conditions:
1—Enzymatic biotransformation by fungi, according to the method described in J. Chem. Soc., Perkin. Trans. 1, 1994, 22, 3319, and in J. Chem. Tech. & Biotech., 1997, 68, 324.
2—Lithium tri-sec-butylborohydride, also known as L-Selectride, lithium borohydride or lithium and aluminium hydride, according to the method described in WO 98/37073.
3—Lithium borohydride and pyridine, according to the method described in Tetrahedron, 2000, 56, 4531, and in WO 98/37073.
4—Magnesium and methanol, according to the method described in WO 2002051823.
5—Catalytic hydrogenation, according to the methods described in EP 306228, in EP 1028960 (improved method) and in WO 01/44240 (improved method).
6—Reaction with sodium dithionite, according to the method described in JP 11049763.

The prior art methods, however, suffer from several drawbacks. The enzymatic biotransformation by fungi requires high dilution and strictly controlled reaction conditions, resulting in a product which is contaminated with considerable amounts of biological substances. The reaction with L-selectride and other boron or aluminium hydrides presents the problem of the high cost and flammability of these reagents. The reaction with lithium borohydride and pyridine is dangerous due to copious evolution of heat and gas. The reaction with magnesium in methanol demands high amounts of magnesium which reacts violently with methanol with evolution of highly flammable and dangerous hydrogen gas. Catalytic hydrogenation involves the use of highly flammable and dangerous hydrogen gas. Furthermore, high amounts of very expensive palladium-based catalysts are necessary for the hydrogenation of Rosiglitazone and related compounds, together with extended reaction times. Finally, the reaction with sodium dithionite is carried out under basic conditions, which lead to a high amount of impurities due to side-reactions.

Thus, it was an object of the present invention to provide a new process for the preparation of Rosiglitazone (I), wherein the drawbacks as indicated above are at least partially eliminated. More particularly, the use of highly flammable reagents such as boron or aluminum hydrides or gaseous hydrogen or the use of expensive noble metal catalysts should be avoided. Further, the process should be easy to scale up and result in high yields of Rosiglitazone with high purity.

Several compounds with a 5-benzyl-2,4-thiazolidinedione structure have been synthesized by reaction of their corresponding 5-benzylidene-2,4-thiazolidinediones, similar to compound (II), with 1,4-dihydropyridines, according to the processes described in J. Org. Chem., 1992, 57, 4047; Tetr. Lett., 1994, 35, 6971 and Tetrahedron: Asymm. 1996, 7, 2515. However, in Tetrahedron, 2000, 56, 4531 it is described that the efficient synthesis of Rosiglitazone (I) is not possible by reaction of 5-{4-[2-(N-methyl-N-(2-pyridyl)amino)ethoxy]benzylidene-2,4-thiazolidinedione (II) with 3,5-dicarbethoxy-2,6-dimethyl-1,4-dihydropyridine—also known as Hantzsch ester. The authors state: “A range of other reduction conditions were investigated including Hantzsch ester and cobalt hydride methods, but gave mostly inefficient or unselective reductions where products derived from both 1,2- and 1,4-reductions were obtained”.

Contrary to what is expected from the state of the art described in the previous references and, particularly, in Tetrahedron, 2000, 56, 4531, we have found that, unexpectedly, Rosiglitazone (I) can be efficiently obtained by reacting 5-{4-[2-(N-methyl-N-(2-pyridyl)amino)ethoxy]benzylidene-2,4-thiazolidinedione (II) with Hantzsch ester and other 1,4-dihydropyridines as the present invention describes.

Thus, a first aspect of the invention provides a process for the preparation of 5-{4-[2-(N-methyl-N-(2-pyridyl)amino)ethoxy]benzyl-2,4-thiazolidinedione (Rosiglitazone) (I)

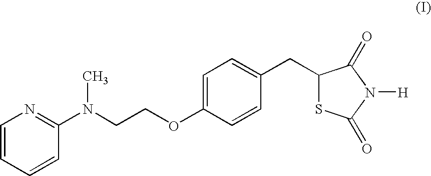

or a pharmaceutically acceptable salt, solvate, hydrate or clathrate thereof, comprising reacting 5-{4-[2-(N-methyl-N-(2-pyridyl)amino)ethoxy]benzylidene-2,4-thiazolidinedione (II)

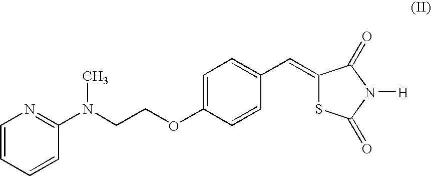

with a 1,4-dihydropyridine (III)

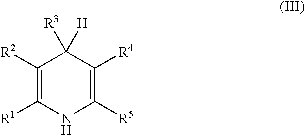

wherein R¹, R², R³, R⁴ and R⁵ are each independently selected from H, halo, OY, NY¹Y², linear or branched or cyclic alkyl, aryl, heteroaryl or COX,

Y is linear or branched or cyclic alkyl, aryl, heteroaryl or COX,

Y¹ and Y² are each independently H, linear, branched or cyclic alkyl, aryl, heteroaryl or COX,

X is H, linear, branched or cyclic alkyl, aryl, heteroaryl, OZ, or NZ¹Z², and

Z, Z¹ and Z² are H, linear, branched or cyclic alkyl, aryl or heteroaryl.

In formula (III) halo stands for F, Cl, Br, or I. Linear or branched alkyl radicals have usually 1 to 6, preferably 1 to 4 carbon atoms. Cyclic alkyl radicals preferably have 3 to 8 carbon atoms. Aryl radicals preferably are mono- or bicyclic aryl radicals such as phenyl or naphthyl. Heteroaryl radicals preferably are mono- or bicyclic radicals comprising at least one heteroatom selected from N, O or S.

In the 1,4-dihydropyridines (III) the substituents R² and R⁴ are preferably COX, wherein X is as defined above. More preferably, R² and R⁴ are COOZ, wherein Z is linear alkyl, particularly methyl or ethyl. R³ is preferably H. R¹ and R⁵ are preferably alkyl, particularly methyl.

The most preferred 1,4-dihydropyridines are 3,5-dicarbethoxy-2,6-dimethyl-1,4-dihydropyridine (Hantzsch ethyl ester) and 3,5-dicarbomethoxy-2,6-dimethyl-1,4-dihydropyridine (Hantzsch methyl ester).

The reaction of compound (II) to Rosiglitazone is preferably carried out in an organic solvent. Examples of suitable organic solvents are aromatic solvents such as toluene or xylene, ketones such as 4-methyl-2-pentanone, alcohols such as n-butanol, esters such as n-butylacetate and saturated hydrocarbon solvents such as heptane. Aromatic solvents are particularly preferred. It is further preferred to use a solvent which allows azeotropic distillation of any water formed in the course of the reaction.

The reaction is preferably carried out at an elevated temperature of at least 60° C., more preferably at reflux conditions for the respective solvent. The reaction time preferably is from 1 h to 24 h.

Preferably, the reaction of (II) to Rosiglitazone (I) is carried out in the presence of a catalyst, whereby the reaction rate is accelerated. Preferred catalysts are metal oxide catalysts such as catalysts based on aluminum oxide or silicon oxide structures or derivatives such as salts thereof. Especially preferred catalysts are aluminum or silicon oxides or aluminates and/or silicates such as magnesium silicate. Silicon oxide is preferably used in the form of a silica gel.

Further, it is preferred that the reaction of (II) to Rosiglitazone (I) is carried out under anhydrous conditions. More preferably, water removal is effected in situ during the reaction in order to avoid any interaction between water and the reagents. Most preferably, water is removed by azeotropic distillation.

The yield of Rosiglitazone (I) in the process of the invention is preferably at least 30%, more preferably at least 50% and most preferably at least 70% based on the weight of compound (II).

A further aspect of the present invention relates to a sequential implementation of two different chemical reactions which may be carried out in the same recipient without subjecting intermediate products resulting from the first chemical reaction to any workup, separation and/or purification. Thus, a considerable saving of time and financial resources is achieved.

This further aspect provides a process for the preparation of Rosiglitazone (I)

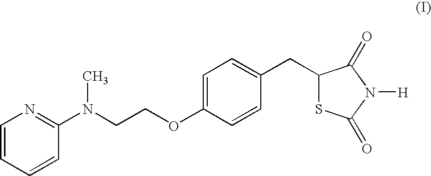

or a pharmaceutically acceptable salt, solvate, hydrate or clathrate thereof, comprising reacting 4-[2-(N-methyl-N(2-pyridyl)amino)ethoxy]benzaldehyde (IV)

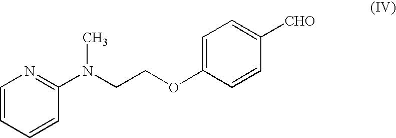

with thiazolidine-2,4-dione (V)

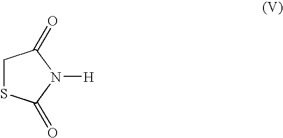

yielding the intermediate 5-{4-[2-(N-methyl-N-(2-pyridyl)amino)ethoxy]benzylidene-2,4-thiazolidinedione (II)

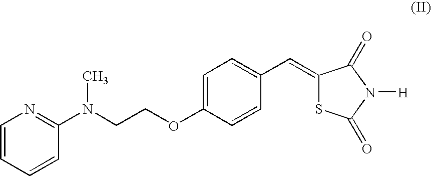

which is reacted with a 1,4-dihydropyridine (III)

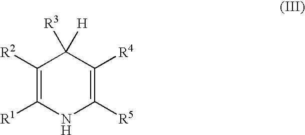

wherein R¹, R², R³, R⁴ and R⁵ are each independently selected from H, halo, OY, NY¹Y², linear or branched or cyclic alkyl, aryl, heteroaryl or COX,

Y is linear or branched or cyclic alkyl, aryl, heteroaryl or COX,

Y¹ and Y² are each independently H, linear, branched or cyclic alkyl, aryl, heteroaryl or COX,

X is H, linear, branched or cyclic alkyl, aryl, heteroaryl, OZ, or NZ¹Z², and

Z, Z¹ and Z² are H, linear, branched or cyclic alkyl, aryl or heteroaryl.

Particularly, this aspect also relates to a two-step process for the preparation of Rosiglitazone (I), whereby the following operations are sequentially carried out inside the same recipient:
(1) The preparation of 5-{4-[2-(N-methyl-N-(2-pyridyl)amino)ethoxy]benzylidene-2,4-thiazolidinedione of formula (II) by condensation of 4-[2-(N-methyl-N-(2-pyridyl)amino)ethoxy]benzaldehyde of formula (IV) with thiazolidine-2,4-dione of formula (V).
(2) The conversion of 5-{4-[2-(N-methyl-N-(2-pyridyl)amino)ethoxy]benzylidene-2,4-thiazolidinedione of formula (II) into Rosiglitazone (I) by reaction with a 1,4-dihydropyridine having the general formula (III) as described above.

The first step of the procedure, the reaction of (IV) with (V), is carried out in an organic solvent, preferably under anhydrous conditions. More preferably, a water removal is carried out in situ during the reaction, preferably by azeotropic distillation.

The solvent which is used for this reaction step preferably is an aromatic solvent, particularly toluene or xylene, i.e. the solvent which is also preferably used in the second reaction step.

Further, it is preferred that the first reaction step is carried out in the presence of a catalyst which may be an ammonium salt, e.g. a pyrrolidinium salt, more preferably pyrrolidinium acetate.

For the second step of the procedure, it is preferred that the intermediate (II), which is obtained after the first reaction step, is reacted further without workup, separation and/or purification, especially in the same recipient where the first step has taken place.

Further, all preferred features as indicated above for the first aspect of the present invention also apply to the second step of the two-step process.

The present invention shall be further illustrated by the Examples given below, however, without limiting the scope of what is regarded as the invention.

### EXAMPLES

### Example 1

Preparation of Rosiglitazone (I) by reaction of 5-{4-[2-(N-methyl-N-(2-pyridyl)amino)ethoxy]benzylidene-2,4-thiazolidinedione of formula (II) with 3,5-dicarbethoxy-2,6-dimethyl-1,4-dihydropyridine (III) in toluene at reflux temperature, using silica gel as catalyst and removing water by azeotropic distillation.

A mixture of 50.0 g (141 mmols) of 5-{4-[2-(N-methyl-N-(2-pyridyl)amino)ethoxy]benzylidene-2,4-thiazolidinedione (II), 71.3 g (281 mmols) of 3,5-dicarbethoxy-2,6-dimethyl-1,4-dihydropyridine (Hantzsch ethyl ester), 30.0 g of silica gel and 300 mL of toluene are stirred under nitrogen atmosphere at reflux temperature for 12 hours with azeotropic distillation of water. After cooling to room temperature, the resulting solid, containing the product and silica gel, is filtered. Silica gel is removed by digestion of the solid with tetrahydrofuran and filtration. The solvent is removed by distillation and the resulting crude residue is purified by crystallization from n-butyl acetate, yielding 33.7 g (67%) of Rosiglitazone (I).

### Example 2

Preparation of Rosiglitazone (I) by an analogous process to that described in Example 1, wherein xylene is used as solvent instead of toluene, yielding 76% of Rosiglitazone (I).

### Example 3

Preparation of Rosiglitazone (I) by an analogous process to that described in Example 1, wherein 4-methyl-2-pentanone is used as solvent instead of toluene, yielding 35% of Rosiglitazone (I).

### Example 4

Preparation of Rosiglitazone (I) by an analogous process to that described in Example 1, wherein n-butanol is used as solvent instead of toluene, yielding 43% of Rosiglitazone (I).

### Example 5

Preparation of Rosiglitazone (I) by an analogous process to that described in Example 1, wherein n-butyl acetate is used as solvent instead of toluene, yielding 50% of Rosiglitazone (I).

### Example 6

Preparation of Rosiglitazone (I) by an analogous process to that described in Example 1, wherein heptane is used as solvent instead of toluene, yielding 39% of Rosiglitazone (I).

### Example 7

Preparation of Rosiglitazone (I) by an analogous process to that described in Example 1, wherein 3,5-dicarbomethoxy-2,6-dimethyl-1,4-dihydropyridine (Hantzsch methyl ester) is used as reducing agent instead of 3,5-dicarbethoxy-2,6-dimethyl-1,4-dihydropyridine, yielding 61% of Rosiglitazone (I).

### Example 8

Preparation of Rosiglitazone (I) by means of two sequential processes carried out into the same recipient, without isolation of the intermediate 5-{4-[2-(N-methyl-N-(2-pyridyl)amino)ethoxy]benzylidene-2,4-thiazolidinedione (II).

A mixture of 20.0 g (78 mmol) of 4-[2-(N-methyl-N-(2-pyridyl)amino)ethoxy]benzaldehyde (IV), 9.6 g (82 mmol) of thiazolidine-2,4-dione (V), 0.3 mL (3.9 mmol) of pyrrolidine, 0.2 mL (3.9 mmol) of glacial acetic acid and 120 mL of toluene is stirred under nitrogen atmosphere at reflux temperature with azeotropic distillation of water for two hours. After cooling to room temperature, 39.5 g (156 mmol) of 3,5-dicarbethoxy-2,6-dimethyl-1,4-dihydropyridine (Hantzsch ethyl ester), 13.9 g of silica gel and 46 mL of toluene are added. The resulting suspension is heated to reflux temperature with azeotropic distillation of water for 13 hours. The reaction is cooled to room temperature, and the resulting solid is filtered. Silica gel is removed after digestion with tetrahydrofuran and filtration of the solid. The solvent is removed by distillation delivering a crude material that is purified by crystallization from n-butyl acetate, yielding 16.3 g (58%) of Rosiglitazone (I).

1. A process for the preparation of 5-{4-[2-(n-methyl-n-(2-pyridyl)amino)ethoxy]benzyl-2,4-thiazolidinedione (Rosiglitazone) (I) 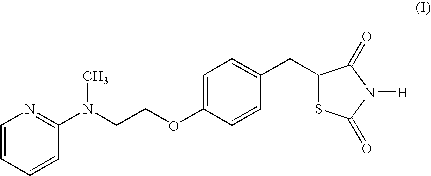
or a pharmaceutically acceptable salt, solvate, hydrate or clathrate thereof, comprising reacting 5-{4-[2-(N-methyl-N-(2-pyridyl)amino)ethoxyl]benzylidene-2,4-thiazolidinedione (II) 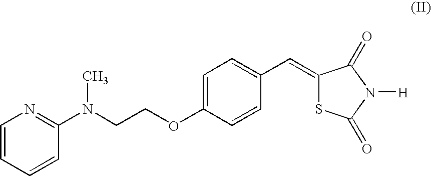
with a 1,4-dihydropyridine (III) 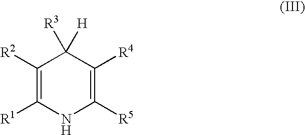
wherein R¹, R², R³, R⁴ and R⁵ are each independently selected from H, halo, OY, NY¹Y², linear or branched or cyclic alkyl, aryl, heteroaryl or COX,
Y is linear or branched or cyclic alkyl, aryl, heteroaryl or COX,
Y¹ and Y² are each independently H, linear, branched or cyclic alkyl, aryl, heteroaryl or COX,
X is H, linear, branched or cyclic alkyl, aryl, heteroaryl, OZ, or NZ¹Z², and
Z, Z¹ and Z² are H, linear, branched or cyclic alkyl, aryl or heteroaryl.

2. The process of claim 1, wherein the reaction is carried out in an organic solvent selected from aromatic solvents, ketones, alcohols, esters and saturated hydrocarbons.

3. The process of claim 2, wherein the reaction is carried out in an aromatic solvent, particularly toluene or xylene.

4. The process of claim 1, wherein R³ and R⁴ are COX.

5. The process of claim 1, wherein R³ and R⁴ are COOZ, wherein Z is linear or branched alkyl, particularly methyl or ethyl.

6. The process of claim 1, wherein R³ is H.

7. The method of claim 1, wherein R¹ and R⁵ are alkyl, particularly methyl.

8. The method of claim 1, wherein the 1,4-dihydropyridine (III) is selected from 3,5-dicarboethoxy-2,6-dimethyl-1,4-dihydropyridine (Hantzsch ethyl ester) and 3,5-dicarbomethoxy-2,6-dimethyl-1,4-dihydropyridine (Hantzsch methyl ester).

9. The method of claim 1, wherein the reaction is carried out in the presence of a catalyst.

10. The method according to claim 9, wherein the catalyst is selected from aluminum oxide, silicon oxide or derivatives thereof.

11. The method according to claim 10, wherein the catalyst is silicon oxide.

12. The method according to claim 1, wherein water removal is carried out during the reaction.

13. The method according to claim 12, wherein water is removed by azeotropic distillation.

14. The method according to claim 1, wherein the yield of compound (I) is at least 30%.

15. A process for the preparation of Rosiglitazone (I) 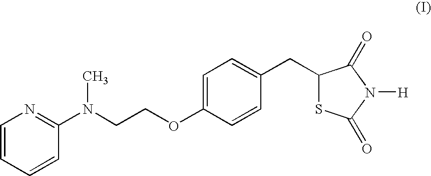
or a pharmaceutically acceptable salt, solvate, hydrate or clathrate thereof, comprising reacting 4-[2-(N-methyl-N(2-pyridyl)amino)ethoxy]benzaldehyde (IV) 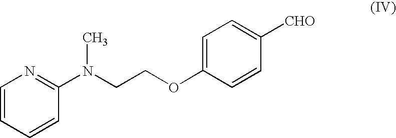
with thiazolidine-2,4-dione (V) 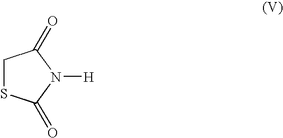
yielding the intermediate 5-{4-[2-(N-methyl-N-(2-pyridyl)amino)ethoxy]-benzylidene-2,4-thiazolidinedione (II) 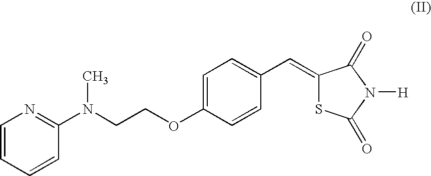
which is reacted with a 1,4-dihydropyridine (III) 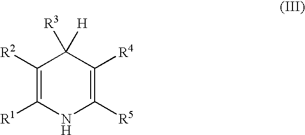
wherein R¹, R², R³, R⁴ and R⁵ are each independently selected from H, halo, OY, NY¹Y², linear or branched or cyclic alkyl, aryl, heteroaryl or COX,
Y is linear or branched or cyclic alkyl, aryl, heteroaryl or COX,
Y¹ and Y² are each independently H, linear, branched or cyclic alkyl, aryl, heteroaryl or COX,
X is H, linear, branched or cyclic alkyl, aryl, heteroaryl, OZ, or NZ¹Z², and
Z, Z¹ and Z² are H, linear, branched or cyclic alkyl, aryl or heteroaryl.

16. The process of claim 15, wherein water removal is carried out during the first reaction step.

17. The process of claim 16, wherein water is removed by azeotropic distillation.

18. The process of claim 15, wherein the reaction of compounds (IV) and (V) to compound (II) is carried out in an aromatic solvent, particularly toluene or xylene.

19. The process according to claim 15, wherein the first reaction step is carried out in the presence of a catalyst.

20. The method of claim 19, wherein the catalyst is an ammonium salt.

21. The process of claim 20, wherein the catalyst is a pyrrolidinium salt, particularly pyrrolidinium acetate.

22. The process according to claim 15, wherein the intermediate (II) is reacted without work-up, separation and/or purification.

23. The process of claim 23, wherein both reaction step1 are carried out in the same recipient.
